(19) United States
(12) Patent Application Publication
(10) Pub. No.: US 2025/0135228 A1
(43) Pub. Date: May 1, 2025

(54) PREDICTION SUPPORT SYSTEM, PREDICTION SUPPORT METHOD, AND PREDICTION SUPPORT PROGRAM

(71) Applicants: OSAKA UNIVERSITY, Osaka (JP); MIZUHO RESEARCH & TECHNOLOGIES, LTD., Tokyo (JP)

(72) Inventors: Teiji NISHIO, Suita shi (JP); Yuya NEMOTO, Tokyo (JP); Hidemasa MAEKAWA, Tokyo (JP)

(21) Appl. No.: 18/837,275

(22) PCT Filed: Feb. 15, 2023
(86) PCT No.: PCT/JP2023/005197

§ 371 Date: Aug. 9, 2024

(30) Foreign Application Priority Data
Feb. 16, 2022 (JP) 2022-022357

(51) Int. Cl. A61N 5/10; G16H 20/40

(52) U.S. Cl. CPC A61N 5/103; A61N 5/1071; G16H 20/40; A61N 2005/1087

(57) ABSTRACT

A circuitry of a prediction support system calculates, through simulation, a first three-dimensional activity distribution and measurement information. The first three-dimensional activity distribution is obtained by changing, with respect to a reference dose distribution obtained when particle beams are radiated on a patient state in an irradiation state based on an irradiation condition of the particle beams, at least one of the irradiation state and the patient state. The measurement information is obtained by two-dimensionally measuring the first three-dimensional activity distribution. The circuitry generates a prediction model for predicting the first three-dimensional activity distribution from the measurement information, and in a case in which the particle beams are radiated based on the irradiation condition of the particle beams and a measurement result of an actual two-dimensional measurement is acquired, applies the prediction model to the measurement result to predict a second three-dimensional activity distribution corresponding to the measurement result.

### TECHNICAL FIELD

The present disclosure relates to a prediction support system, a prediction support method, and a prediction support program for supporting therapy using particle beams.

### BACKGROUND ART

When a therapy is performed using radiation, it is necessary to apply an appropriate amount of radiation to an accurate position in order to reduce the exposure dose of a patient. In this regard, a radiotherapy control device has been developed that calculates the current positions of markers implanted in a patient to reduce the patient's exposure dose to imaging radiation emitted from fluoroscopic imaging devices used during radiotherapy (for example, refer to Patent Literature 1). The technology described in this document acquires fluoroscopic images of three or more markers from a set of fluoroscopic imaging devices and acquires the distances between the markers. Then, by calculating the current position of each marker, it is determined whether or not to irradiate the therapeutic radiation.

In some cases, therapy is performed by using high-energy and high-speed particle beams. A proton beam, which is a type of particle beam, loses a large amount of energy at a location immediately before the incident protons stop inside the body. At this location, a high dose region called the “Bragg peak” is formed. Thus, by reducing damages to normal regions, it is possible to intensively irradiate an affected part in the body with strong radiation. With regard to such irradiation of particle beams (proton beams), a technique has been developed that uses the nuclear spallation reaction occurring between incident protons and target nuclei within the patient's body to visualize the area irradiated with the proton beams (hereafter, the “irradiation area”) and derive the irradiation dose for the tumor from this visualization information (for example, see Non-Patent Literature 1). The technology described in this document uses a positron emission tomography device (PET device) called the “beam on-line positron emission tomography system”, which detects positron-emitting nuclei generated in the irradiation area within the patient's body through the target nuclear spallation reaction in proton beam therapy. This PET device is installed in a proton beam rotating gantry irradiation chamber to visualize a proton beam irradiation region.

### CITATION LIST

### Patent Literature

Patent Literature 1: Japanese Laid-Open Patent Publication No. 2013-192702

### Non-Patent Literature

Non-Patent Literature 1: Nishio Teiji, “Innovative proton therapy with irradiated volume image by target nuclear fragment reaction”, [online], 2011, Research Organization for Information Science and Technology, RIST News, No. 50, p. 24-35, [Retrieved on Dec. 24, 2021], Internet <URL:http://www.rist.or.jp/rnews/50/50s4.pdf>

### SUMMARY OF INVENTION

### Technical Problem

However, in the above-described positron emission tomography apparatus, an activity distribution, which is a three-dimensional distribution, is measured two-dimensionally. Therefore, it may be difficult to check the three-dimensional dose distribution.

### Solution to Problem

In an aspect, a prediction support system is provided. The prediction support system comprises a control unit that predicts an irradiation state of a particle beam irradiation device. The control unit is configured to calculate, through simulation, a first three-dimensional activity distribution and measurement information, the first three-dimensional activity distribution being obtained by changing, with respect to a reference dose distribution obtained when particle beams are radiated on a patient state in an irradiation state based on an irradiation condition of the particle beams, at least one of the irradiation state and the patient state, and the measurement information being obtained by two-dimensionally measuring the first three-dimensional activity distribution, generate a prediction model for predicting the first three-dimensional activity distribution from the measurement information, and in a case in which the particle beams are radiated based on the irradiation condition of the particle beams and a measurement result of an actual two-dimensional measurement is acquired, apply the prediction model to the measurement result to predict a second three-dimensional activity distribution corresponding to the measurement result.

In another aspect, a method for predicting an irradiation state of a particle beam irradiation device using a prediction support system including a control unit is provided. The method comprises the control unit calculating, through simulation, a first three-dimensional activity distribution and measurement information, the first three-dimensional activity distribution being obtained by changing, with respect to a reference dose distribution obtained when particle beams are radiated on a patient state in an irradiation state based on an irradiation condition of the particle beams, at least one of the irradiation state and the patient state, and the measurement information being obtained by two-dimensionally measuring the first three-dimensional activity distribution, the control unit generating a prediction model for predicting the first three-dimensional activity distribution from the measurement information, and the control unit, in a case in which the particle beams are radiated based on the irradiation condition of the particle beams and a measurement result of an actual two-dimensional measurement is acquired, applying the prediction model to the measurement result to predict a second three-dimensional activity distribution corresponding to the measurement result.

In yet another aspect, a program for predicting an irradiation state of a particle beam irradiation device using a prediction support system including a control unit is provided. When the program is executed, the control unit is configured to calculate, through simulation, a first three-dimensional activity distribution and measurement information, the first three-dimensional activity distribution being obtained by changing, with respect to a reference dose distribution obtained when particle beams are radiated on a patient state in an irradiation state based on an irradiation condition of the particle beams, at least one of the irradiation state and the patient state, and the measurement information being obtained by two-dimensionally measuring the first three-dimensional activity distribution, generate a prediction model for predicting the first three-dimensional activity distribution from the measurement information, and in a case in which the particle beams are radiated based on the irradiation condition of the particle beams and a measurement result of an actual two-dimensional measurement is acquired, apply the prediction model to the measurement result to predict a second three-dimensional activity distribution corresponding to the measurement result.

### Advantageous Effects of Invention

According to the present disclosure, it is possible to accurately evaluate the irradiation state of particle beams and support a therapy that uses particle beam irradiation.

### BRIEF DESCRIPTION OF DRAWINGS

FIG. 1 is an explanatory diagram illustrating a prediction support system according to an embodiment.

FIG. 2 is an explanatory diagram illustrating a hardware configuration according to the embodiment.

FIG. 3 is an explanatory diagram illustrating an irradiation position of particle beams according to the embodiment.

FIG. 4 is an explanatory diagram illustrating an activity distribution measured two-dimensionally according to the embodiment.

FIG. 5 is an explanatory diagram illustrating a relationship between a three-dimensional activity distribution and a two-dimensional measurement distribution according to the embodiment.

FIG. 6 is an explanatory diagram illustrating a procedure of processes according to the embodiment.

FIG. 7 is an explanatory diagram illustrating a procedure of processes of a learning process according to the embodiment.

FIG. 8 is an explanatory diagram illustrating training information according to the embodiment.

FIG. 9 is an explanatory diagram of an adjustment coefficient according to the embodiment.

FIG. 10 is an explanatory diagram illustrating a procedure of processes of a prediction process according to the embodiment.

### DESCRIPTION OF EMBODIMENTS

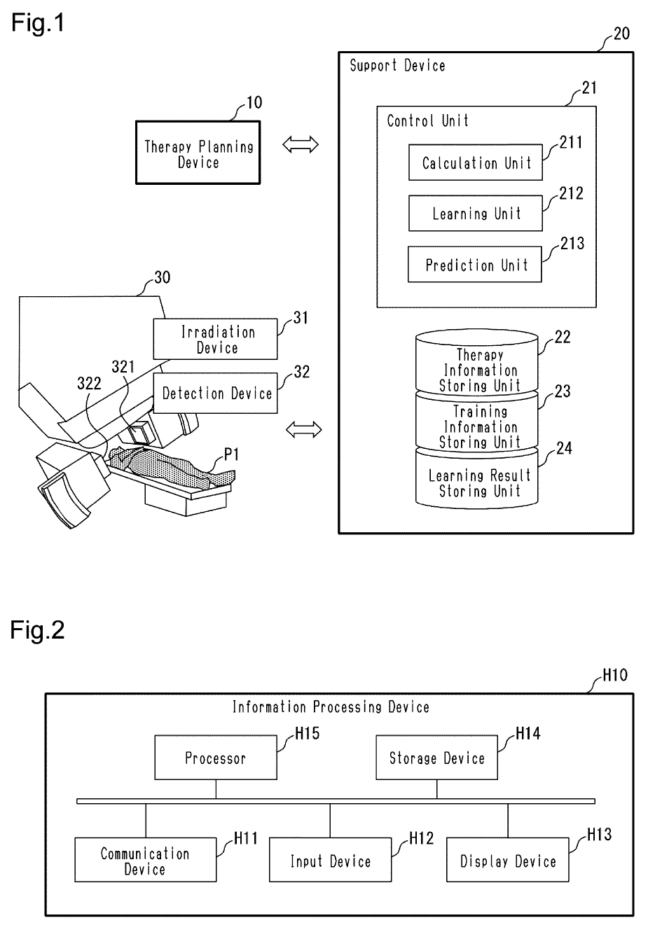

A prediction support system, a prediction support method, and a prediction support program according to an embodiment will be described with reference to FIGS. 1 to 10. In the present embodiment, a case will be described in which an affected part of a patient is irradiated with protons as particle beams to treat the affected part.

In the present embodiment, a therapy planning device 10, a support device 20, and a therapy device 30, which are connected via a network, are used.

### Example of Hardware Configuration

FIG. 2 shows an example of hardware configuration example of an information processing device H10, which functions as the therapy planning device 10, the support device 20, the therapy device 30, and the like.

The information processing device H10 includes a communication device H11, an input device H12, a display device H13, a storage device H14, and a processor H15. This hardware configuration is merely one example, and other hardware may be employed.

The communication device H11 is an interface that establishes communication paths with other devices so as to transmit and receive data. The communication device H11 is, for example, a network interface or a wireless interface.

The input device H12 receives input from a user, and is, for example, a mouse or a keyboard. The display device H13 is, for example, a display or a touch screen that displays various types of information.

The storage device H14 stores data and various programs to implement the functionalities of the therapy planning device 10, the support device 20, and the therapy device 30. Examples of the storage device H14 include non-transitory computer readable media such as a ROM, a RAM, and a hard disk.

The processor H15 uses programs and data stored in the storage device H14, so as to control processes in the therapy planning device 10, the support device 20, and the therapy device 30 (for example, processes in a control unit 21, which will be discussed below). Examples of the processor H15 include a CPU and an MPU. The processor H15 expands, in RAM, programs stored in ROM or the like, so as to execute various processes. For example, when an application program of the therapy planning device 10, the support device 20, and the therapy device 30 is started, the processor H15 operates to execute processes discussed below.

The processor H15 does not have to execute all processes through software-processing. For example, the processor H15 may include a dedicated hardware circuit, such as an application specific integrated circuit (ASIC) that executes at least some processes through hardware-processing. More specifically, the processor H15 may be any of the following.

[1] One or more processors that operate according to a computer program.

[2] One or more dedicated hardware circuits that execute at least part of various processes.

[3] Circuitry including a combination of the above elements.

The processor includes a central processing unit (CPU) and memories such as a random-access memory (RAM) and a read-only memory (ROM). The memories store program codes or commands configured to cause the CPU to execute processes. Memory or computer-readable media includes any available media that can be accessed by a general purpose or special purpose computer.

### Functions of Therapy Planning Device 10, Support Device 20, and Therapy Device 30

Next, functions of the therapy planning device 10, the support device 20, and the therapy device 30 will be described.

The therapy planning device 10 is a simulator for examining a method of emitting radiation to an affected area and checking whether an appropriate dose is being provided. The therapy planning device 10 acquires CT images (DICOM data) obtained by tomographic imaging at specified image intervals from a CT imaging apparatus. Then, the therapy planning device 10 performs contour extraction on the DICOM data by using a known method to generate CT contour information. The CT contour information is constituted by DICOM region of interest (ROI) data, which includes an aggregate of points (coordinates) constituting a contour of a specified site (a body surface, a bone, an affected part, an organ at risk, or the like) that is identified in CT images (tomographic images) captured at specified intervals. Based on the surface shape of the affected area, the shape of the affected area, its position, and the positional relationship with organs at risk, the therapy planning device 10 determines parameters such as therapy beam quality, its angle of incidence, an irradiation range, and the number of times of irradiation of prescribed dose.

The support device 20 is a computer system for supporting particle beam (proton beam) therapy. The support device 20 includes a control unit 21, a therapy information storing unit 22, a training information storing unit 23, and a learning result storing unit 24.

The control unit 21 executes processes discussed below (processes including a calculation stage, a learning stage, and a prediction stage). When executing the prediction support program for these stages, the control unit 21 functions as a calculation unit 211, a learning unit 212, a prediction unit 213, and the like.

The calculation unit 211 executes a process of creating training information used for machine learning through simulation. The calculation unit 211 stores in advance change factors for adjusting a three-dimensional activity distribution. As the change factors, for example, a three-dimensional dose distribution and an image processing coefficient for finely correcting a three-dimensional CT image can be used.

The learning unit 212 executes a process of calculating an adjustment coefficient through machine learning.

The prediction unit 213 executes a process of predicting the irradiation dose using the adjustment coefficient.

The therapy information storing unit 22 stores therapy management information on proton beam irradiation for therapy of a patient. When the support device 20 acquires therapy plan information from the therapy planning device 10, the therapy information storing unit 22 stores the acquired therapy management information. The therapy management information includes CT contour information and irradiation condition information in association with a patient code and a scheduled therapy date.

The patient code is an identifier for identifying each patient.

The scheduled therapy date is a scheduled date (year, month, and day) of therapy with proton beam irradiation in the therapy plan for this patient.

The CT contour information includes, as a patient state, position information of the contour of a specified site (a body surface, a bone, an affected part, an organ at risk, or the like) in CT images of the affected part of the patient.

The irradiation condition information is a condition for irradiating this patient with proton beams on the scheduled therapy date. The irradiation state (three-dimensional dose distribution and three-dimensional activity distribution) is determined by the irradiation condition. The irradiation condition information includes information on the irradiation position, an irradiation direction, an irradiation energy, an irradiation dose, a beam irradiation method, and the like of the proton beams. Examples of the beam irradiation method include a “spread-out beam irradiation method” and a “scanning irradiation method”.

The training information storing unit 23 stores training information for calculating an adjustment coefficient through machine learning. When the learning process is performed, the training information storing unit 23 stores training information. The training information includes simulation information for each patient code.

The patient code is an identifier for identifying each patient.

The simulation information includes the following.

Three-Dimensional Activity Distribution (Simulation) without Change Factors

This three-dimensional activity distribution is calculated through a simulation in which a CT image region of a patient is irradiated with particle beams based on a therapy plan.

Three-Dimensional Activity Distribution (Simulation) with Change Factors Added

This three-dimensional activity distribution is calculated through simulation in which change factors are generated. Examples of the change factors include a change in an irradiation condition (a range of quantum beams or the like) and a change in a CT image region (a change in the shape of an affected part such as a tumor or a movement of the patient).

Two-Dimensional Measurement Distribution (Simulation) with Change Factors Added

The two-dimensional measurement distribution is calculated on the assumption that a three-dimensional activity distribution with change factors is measured on a two-dimensional plane.

The learning result storing unit 24 stores adjustment coefficient information for calculating an irradiation dose. When the learning process is performed, the learning result storing unit 24 stores the adjustment coefficient information. The adjustment coefficient information includes information on the patient code and the adjustment coefficient.

The patient code is an identifier for identifying each patient.

The adjustment coefficient is a prediction model for predicting a three-dimensional irradiation dose from an activity distribution in a body part of a patient.

The therapy device 30 is a device that treats an affected area such as cancer by irradiating the affected area with radiation. The therapy device 30 is equipped with a treatment table that allows a patient P1 to maintain a therapy posture (such as supine or prone). The therapy device 30 includes an irradiation device 31 and detection devices 32.

The irradiation device 31 is a device (gantry) that irradiates the patient P1 on the treatment table with particle beams.

The detection devices 32 are each a positron emission tomography device (PET device), which detects positron-emitting nuclei that are generated in the irradiation area within the patient's body through the target nuclear spallation reaction in the proton beam therapy. The irradiation depth position (irradiation region) can be identified by the emission position of the positron emission nuclei. The detection devices 32 respectively include measurement planes 321, 322 for detecting positron emission nuclei on the sides of the irradiation direction of the proton beams irradiated from the irradiation device 31.

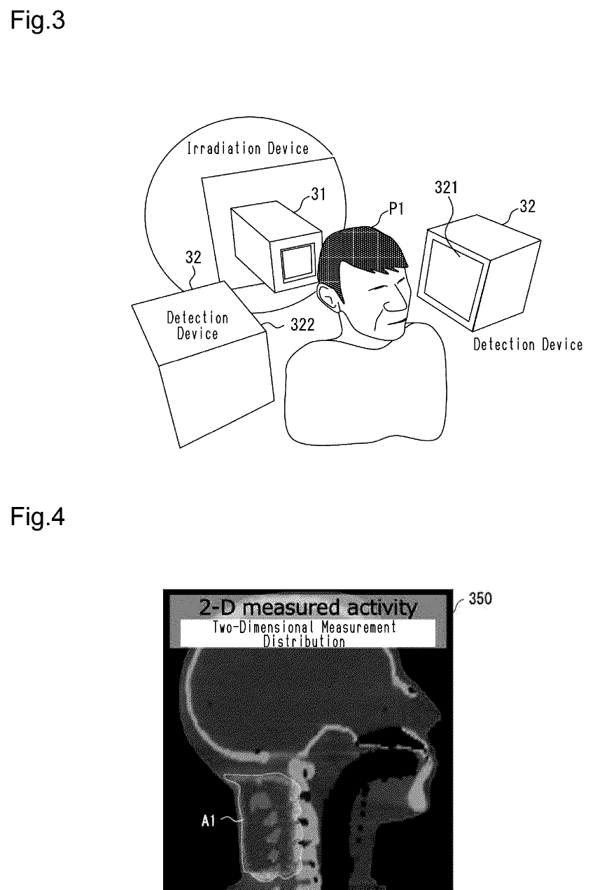

A case in which, for example, the neck of a patient is irradiated with particle beams will be described with reference to FIG. 3. In this case, the particle beams are irradiated from the irradiation device 31 behind the neck of the patient P1. Then, the activity distribution is two-dimensionally measured by the measurement planes 321, 322 of the detection devices 32 arranged on the opposite sides of the head.

In this case, as shown in FIG. 4, an activity distribution A1 of the neck is measured in a two-dimensional measurement image 350.

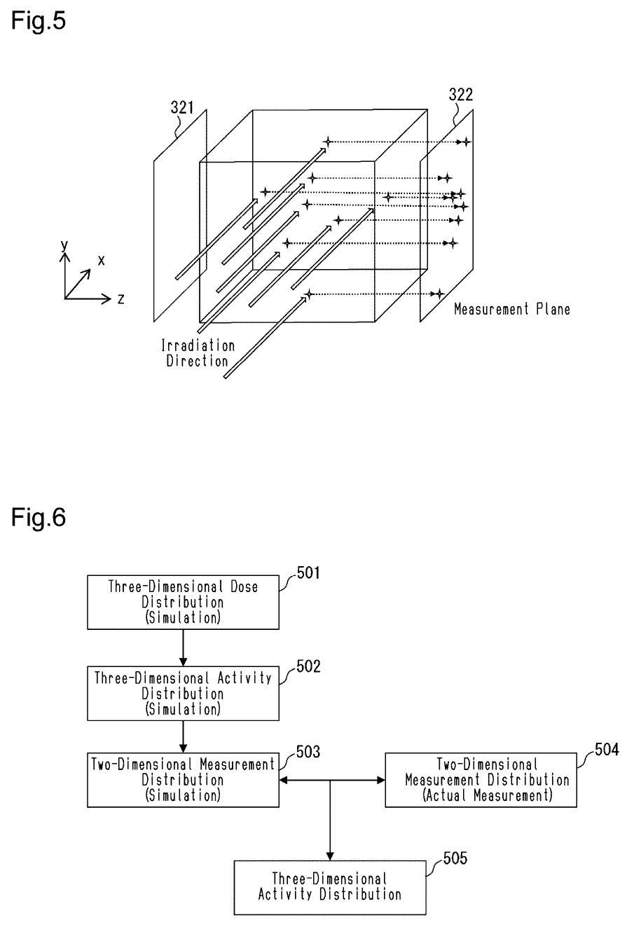

As shown in FIG. 5, activity is generated in a three-dimensional space (x, y, z) in the body by the particle beams irradiated in the x-axis direction. The activity is measured on a measurement plane (x, y) by the flat measurement planes 321, 322. In this measurement plane (x, y), the activity distribution in the z-axis direction is compressed. That is, information on the activity distribution in the z-axis direction may not be partially obtained.

Therefore, as shown in FIG. 6, a three-dimensional dose distribution 501 (reference dose distribution) is calculated based on the therapy plan through simulation. Next, a three-dimensional activity distribution 502 is generated through simulation in accordance with the CT image region of the patient. Then, a two-dimensional measurement distribution 503, which is compressed in the z-axis direction, is calculated. That is, the two-dimensional measurement distribution 503 is calculated by compressing the three-dimensional activity distribution 502 in the z-axis direction. When a two-dimensional measurement distribution 504 is acquired by actual measurement, the calculated two-dimensional measurement distribution 503 corresponds to the two-dimensional measurement distribution 504 acquired by actual measurement.

In this case, the simulation result of the activity distribution of the particle beam irradiation and the actual activity distribution have the following relationship.

$${A}_{3 D - mea .} ( x , y , z ) = {A}_{2 D - mea .} ( x , y ) \times \frac{{A}_{3 D - c al .} ( x , y , z )}{{\sum }_{z} {A}_{3 D - c al .} ( x , y , z )} \qquad [ Expression 1 ]$$

A_{3D-mea}. is an actual three-dimensional activity distribution.

A_{2D-mea}. is a two-dimensional measurement distribution (measurement result) of the actually measured activity.

A_{3D-cal}. is a three-dimensional activity distribution obtained through an irradiation simulation.

A_{2D-cal}. is a two-dimensional activity distribution (measurement information) obtained through an irradiation simulation.

The irradiation simulation and the actual irradiation may deviate from each other depending on the irradiation state and the patient state. In such a case, a deviation occurs between the two-dimensional measurement distribution 503 and the two-dimensional measurement distribution 504.

In this case, adjustment is performed using the following relational expression.

$${A}_{3 D - mea .}^{′} ( x , y , z ) = {A}_{2 D - mea .} ( x , y ) \times \frac{{A}_{3 D - ca l .}^{′} ( x , y , z )}{{\sum }_{z} {A}_{3 D - ca l .}^{′} ( x , y , z )} \\ {A}_{3 D - ca l .}^{′} ( x , y , z ) = F ( x , y , z ) \times {A}_{3 D - ca l .} ( x , y , z ) \\ F = ( \begin{matrix}{f}_{11} & {f}_{12} & {f}_{13} \\ {f}_{21} & {f}_{22} & {f}_{23} \\ {f}_{31} & {f}_{32} & {f}_{33}\end{matrix} ) \qquad [ Expression 2 ]$$

A′_{3D-cal}. is a three-dimensional activity distribution obtained through an irradiation simulation by taking change factors into consideration.

A′_{3D-mea}. is an actual three-dimensional activity distribution with change factors.

F is a coefficient (adjustment coefficient) for adjusting change factors.

Hereinafter, the adjustment coefficient F is calculated through machine learning, and the actual three-dimensional activity distribution 505 is predicted using the adjustment coefficient F at the time of therapy.

### Irradiation Support Process

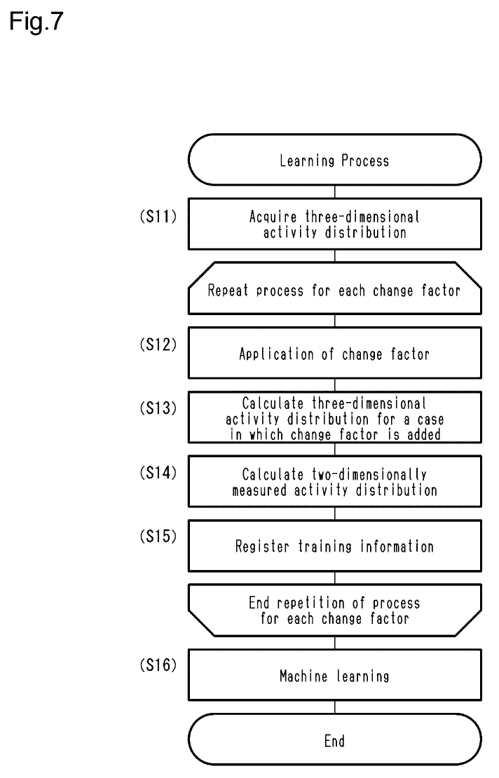

An irradiation support process will be described with reference to FIGS. 7 to 10. The irradiation support process includes a learning process and a prediction process.

### Learning Process

The learning process will be described with reference to FIG. 7.

First, the control unit 21 of the support device 20 performs a process for acquiring a three-dimensional activity distribution (step S11). Specifically, the calculation unit 211 of the control unit 21 acquires a dose distribution for irradiating particle beams based on the therapy plan.

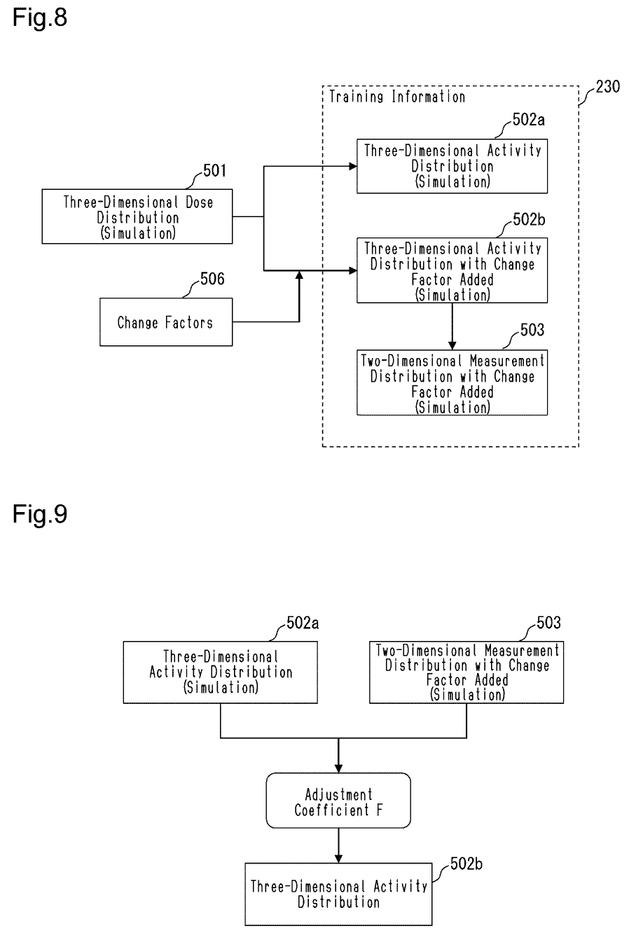

As shown in FIG. 8, a three-dimensional activity distribution 502a is generated through simulation based on the three-dimensional dose distribution 501.

Next, a three-dimensional activity distribution 502b is generated through simulation with a change factor 506 added. Then, a two-dimensional measurement distribution 503 corresponding to the three-dimensional activity distribution 502b is calculated. Training information is thus generated.

Accordingly, the control unit 21 repeats the following process for each change factor.

First, the control unit 21 executes a change factor application process (step S12). Specifically, the calculation unit 211 acquires change factors (a change in the range of the particle beam, a change in the CT image region, and the like) stored in advance. The change in the range of the particle beam is finely adjusted by multiplying the three-dimensional dose distribution 501 by an image processing coefficient of a change factor. The change in the CT image region is finely adjusted by multiplying the CT image of the patient by an image processing coefficient of a change factor.

Next, the control unit 21 executes a process for calculating a three-dimensional activity distribution for a case in which a change factor is added (step S13). Specifically, the calculation unit 211 calculates the three-dimensional activity distribution 502b through simulation including the change factor 506.

Next, the control unit 21 executes a process for calculating a two-dimensionally measured activity distribution (step S14). Specifically, the calculation unit 211 calculates the two-dimensional measurement distribution 503, which is measured on a measurement plane from the calculated three-dimensional activity distribution 502b. The change factor 506 is taken into consideration in the two-dimensional measurement distribution 503.

Next, the control unit 21 executes a process for registering the training information (step S15). Specifically, the learning unit 212 of the control unit 21 generates training information 230 by combining the three-dimensional activity distribution 502a, the three-dimensional activity distribution 502b, in which the change factor 506 is taken into consideration, and the two-dimensional measurement distribution 503, in which change factors are taken into consideration. Then, the learning unit 212 records the generated training information 230 in the training information storing unit 23.

The control unit 21 repeats the above-described processes until the processes are completed for all the change factors.

Then, the control unit 21 executes a machine learning process (step S16). The learning unit 212 calculates the adjustment coefficient F through machine learning using the training information 230 recorded in the training information storing unit 23.

Specifically, as shown in FIG. 9, the learning unit 212 calculates the adjustment coefficient F for predicting the three-dimensional activity distribution 502a based on the three-dimensional activity distribution 502b, which has been generated through a simulation, and the two-dimensional measurement distribution 503, to which change factors have been added. Then, the learning unit 212 records the calculated adjustment coefficient F in the learning result storing unit 24 in association with the patient code.

### Prediction Process

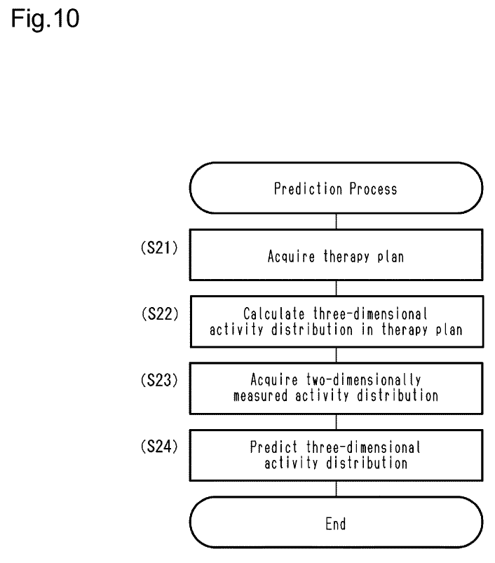

The prediction process will now be described with reference to FIG. 10. This process is executed when the patient is treated with particle beam irradiation.

First, the control unit 21 executes a process for acquiring a therapy plan (step S21). Specifically, the prediction unit 213 of the control unit 21 acquires, from the therapy planning device 10, a therapy plan (an irradiation condition and a CT image) associated with the patient code of the patient to be irradiated with particle beams. Then, the prediction unit 213 calculates a three-dimensional dose distribution using the irradiation condition of the therapy plan.

Next, the control unit 21 executes a process for calculating a three-dimensional activity distribution in the therapy plan (step S22). Specifically, the prediction unit 213 calculates the three-dimensional activity distribution using the three-dimensional dose distribution and the CT image. Variation factors are not taken into consideration in this three-dimensional activity distribution.

Next, the control unit 21 executes a process for acquiring a two-dimensionally measured activity distribution (step S23). Specifically, when irradiation is performed, the prediction unit 213 acquires a two-dimensional measurement distribution from the detection devices 32.

Next, the control unit 21 executes a process for predicting a three-dimensional activity distribution (step S24). Specifically, the prediction unit 213 acquires the adjustment coefficient F associated with the patient code from the learning result storing unit 24. This is the adjustment coefficient F in [Expression 2]. Next, the prediction unit 213 predicts an actual three-dimensional activity distribution by using the acquired adjustment coefficient F, in relation to the acquired two-dimensional measurement distribution and the three-dimensional activity distribution in the therapy plan.

If there is a difference between the three-dimensional activity distribution in the therapy plan and the actual three-dimensional activity distribution, the irradiation condition in the therapy plan is adjusted as necessary.

The present embodiment has the following advantages.

(1) In the present embodiment, the control unit 21 performs the change factor application process (step S12), the process for calculating the three-dimensional activity distribution for a case in which change factors are added (step S13), and the process for calculating the two-dimensionally measured activity distribution (step S14). It is thus possible to generate a two-dimensional measurement distribution for an activity distribution in which change factors are taken into consideration for the irradiation condition of the therapy plan.

(2) In the present embodiment, the control unit 21 executes the machine learning process (step S16). This makes it possible to generate information for predicting the three-dimensional activity distribution from the acquired two-dimensional measurement distribution. In particular, since the influence of the change factors on the entire activity distribution is small, the three-dimensional activity distribution can be efficiently predicted by using the adjustment coefficient F.

(3) In the present embodiment, the control unit 21 performs the process for acquiring the two-dimensionally measured activity distribution (step S23) and the process for predicting the three-dimensional activity distribution (step S24). This allows the irradiation state of particle beams to be checked quickly while performing the therapy. If necessary, the irradiation condition can be changed.

The above-described embodiment may be modified as follows. The above-described embodiment and the following modifications can be combined as long as the combined modifications remain technically consistent with each other.

In the above-described embodiment, proton beams are used as the particle beams. The particle beams are not limited to proton beams, and for example, carbon beams or the like can also be used.

In the above-described embodiment, the detection devices 32 are positron tomography devices, and detect positron emission nuclei generated in the irradiation region in the patient's body. The present disclosure is not limited to detection of positron emission nuclei as long as the irradiation region can be detected.

In the above-described embodiment, the control unit 21 executes the machine learning process (step S16). The control unit 21 calculates the adjustment coefficient F for predicting the three-dimensional activity distribution 502a based on the three-dimensional activity distribution 502b and the two-dimensional measurement distribution 503, to which the change factors have been added. The variables for the input layer and the output layer are not limited these as long as the actual three-dimensional activity distribution can be predicted. For example, the actual three-dimensional activity distribution may be predicted based on the irradiation condition of the therapy plan and the two-dimensional measurement distribution. In the input layer, a two-dimensional measurement distribution calculated through simulation using the dose distribution of the therapy plan may be used.

In the above-described embodiment, the learning result storing unit 24 stores the adjustment coefficient information for calculating the irradiation dose. In this disclosure, a 3×3 matrix is utilized as the adjustment coefficient information. The information used for prediction is not limited to a matrix. For example, a network including an input layer, intermediate layers, and an output layer may be used.

For example, an irradiation state determined from a therapy plan and a two-dimensional activity distribution (measurement result) actually measured two-dimensionally may be used in the input layer. In the output layer, change factors and a three-dimensional activity distribution in which the change factors are taken into consideration are used. Accordingly, it is possible to predict the actual three-dimensional activity distribution and the change factors according to the two-dimensional activity distribution (measurement result).

1. A prediction support system, comprising circuitry that predicts an irradiation state of a particle beam irradiation device, wherein
the circuitry is configured to
calculate, through simulation, a first three-dimensional activity distribution and measurement information, the first three-dimensional activity distribution being obtained by changing, with respect to a reference dose distribution obtained when particle beams are radiated on a patient state in an irradiation state based on an irradiation condition of the particle beams, at least one of the irradiation state and the patient state, and the measurement information being obtained by two-dimensionally measuring the first three-dimensional activity distribution,
generate a prediction model for predicting the first three-dimensional activity distribution from the measurement information, and
in a case in which the particle beams are radiated based on the irradiation condition of the particle beams and a measurement result of an actual two-dimensional measurement is acquired, apply the prediction model to the measurement result to predict a second three-dimensional activity distribution corresponding to the measurement result.

2. The prediction support system according to claim 1, wherein the change in the irradiation state includes a change in a range of the particle beams.

3. The prediction support system according to claim 1, wherein the change in the patient state includes a change in a shape of an affected part of the patient to be irradiated with the particle beams.

4. The prediction support system according to any one of claim 1, wherein the prediction model is a matrix that adjusts the first three-dimensional activity distribution calculated through the simulation to the second three-dimensional activity distribution corresponding to the measurement result.

5. A method for predicting an irradiation state of a particle beam irradiation device using a prediction support system including a circuitry, the method comprising:
the circuitry calculating, through simulation, a first three-dimensional activity distribution and measurement information, the first three-dimensional activity distribution being obtained by changing, with respect to a reference dose distribution obtained when particle beams are radiated on a patient state in an irradiation state based on an irradiation condition of the particle beams, at least one of the irradiation state and the patient state, and the measurement information being obtained by two-dimensionally measuring the first three-dimensional activity distribution,
the circuitry generating a prediction model for predicting the first three-dimensional activity distribution from the measurement information, and
the circuitry, in a case in which the particle beams are radiated based on the irradiation condition of the particle beams and a measurement result of an actual two-dimensional measurement is acquired, applying the prediction model to the measurement result to predict a second three-dimensional activity distribution corresponding to the measurement result.

6. A non-transitory computer readable media storing program for predicting an irradiation state of a particle beam irradiation device using a prediction support system including a circuitry, wherein
the program when executed by the circuitry causing the circuitry to:
calculate, through simulation, a first three-dimensional activity distribution and measurement information, the first three-dimensional activity distribution being obtained by changing, with respect to a reference dose distribution obtained when particle beams are radiated on a patient state in an irradiation state based on an irradiation condition of the particle beams, at least one of the irradiation state and the patient state, and the measurement information being obtained by two-dimensionally measuring the first three-dimensional activity distribution,
generate a prediction model for predicting the first three-dimensional activity distribution from the measurement information, and
in a case in which the particle beams are radiated based on the irradiation condition of the particle beams and a measurement result of an actual two-dimensional measurement is acquired, apply the prediction model to the measurement result to predict a second three-dimensional activity distribution corresponding to the measurement result.

7. The method according to claim 5, wherein the change in the irradiation state includes a change in a range of the particle beams.

8. The method according to claim 5, wherein the change in the patient state includes a change in a shape of an affected part of the patient to be irradiated with the particle beams.

9. The method according to claim 5, wherein the prediction model is a matrix that adjusts the first three-dimensional activity distribution calculated through the simulation to the second three-dimensional activity distribution corresponding to the measurement result.

10. The non-transitory computer readable media according to claim 6, wherein the change in the irradiation state includes a change in a range of the particle beams.

11. The non-transitory computer readable media according to claim 6, wherein the change in the patient state includes a change in a shape of an affected part of the patient to be irradiated with the particle beams.

12. The non-transitory computer readable media according to claim 6, wherein the prediction model is a matrix that adjusts the first three-dimensional activity distribution calculated through the simulation to the second three-dimensional activity distribution corresponding to the measurement result.
